(19) United States
(12) Patent Application Publication
(10) Pub. No.: US 2010/0320404 A1
(43) Pub. Date: Dec. 23, 2010

(54) PARTICLE THERAPY INSTALLATION

(76) Inventor: Eugene Tanke, East Lansing, MI (US)

Correspondence Address: BRINKS HOFER GILSON & LIONE, P.O. BOX 10395, CHICAGO, IL, 60610, US

(21) Appl. No.: 12/918,039

(22) PCT Filed: Jan. 23, 2009
(86) PCT No.: PCT/EP09/50736

§ 371 Date: Aug. 17, 2010

(30) Foreign Application Priority Data
Feb. 25, 2008 (DE) 10 2008 011 015.9

(51) Int. Cl. A61N 5/10

(52) U.S. Cl. 250/492.3

(57) ABSTRACT

A particle therapy system includes an ECR ion source for production of charged ions, which are accelerated in an accelerator unit that follows the ECR ion source. The accelerator unit accelerates the charged ions to an energy that is used for irradiation, where the magnetic fields of the ECR ion source are matched to operation of the ECR ion source for lightweight ions, such that the ECR ion source is operated in the afterglow mode. In the afterglow mode, an afterglow beam pulse is emitted from the ECR ion source after a microwave resonance pulse has been switched off. The current level of the afterglow beam pulse is higher than a current that is emitted from the ECR ion source during use of the microwave resonance pulse.

The present patent document is a §371 nationalization of PCT Application Serial Number PCT/EP2009/050736, filed on Jan. 23, 2009, designating the United State, which is hereby incorporated by reference. This patent document also claims the benefit of DE 10 2008 011 015.9, filed Feb. 25, 2008, which is also hereby incorporated by reference.

### BACKGROUND

The present embodiments relate to a particle therapy system.

Particle therapy is an established method for the treatment of tissue (e.g., tumoral diseases) and usually involves charged particles being accelerated to high energies, formed into a particle beam and directed via a high energy beam transport system into one or a plurality of irradiation chambers. Particle therapy has the advantage that the particle beam interacts with the tissues to be irradiated in a relatively small, confined area so that damage to surrounding tissue that is not to be irradiated can be avoided effectively. In addition to protons, helium ions or pions, other lightweight ions (e.g., ions having a nuclear charge of less than or equal to 10) such as, for example, carbon ions and oxygen ions are also used in particle therapy as particles for irradiation.

Such ions can be generated, for example, by an electron cyclotron resonance (ECR) ion source. An ECR ion source contains plasma enclosed in a magnetic field. The magnetic field includes an axial component and a radial component and is created in such a way that plasma electrons that move on spiral paths around the magnetic field lines due to the Lorentz force can be accelerated to high energies by being bombarded with microwave irradiation. This involves the microwave irradiation being attuned to the magnetic fields in the plasma in order to have a resonant effect on the plasma electrons. The plasma electrons, heated by microwave irradiation, in turn, ionize atoms and/or ions that are further ionized. The ions generated in the plasma can be extracted from the ECR ion source using a high voltage. During bombardment with microwave irradiation, an ion current is consequently emitted from the ECR ion source.

When operating an ECR ion source, the ion current that is emitted often decreases as time goes by. In the case of carbon ions, for example, the decrease in current can be attributed to deposits of atoms of the plasma gas that collect on the walls of the ECR ion source in the course of operation. In order to ensure an adequate ion current and consequently, safe operation of the particle therapy system, the ECR ion source is appropriately dimensioned or serviced at appropriately frequent intervals, which leads to overall higher costs and greater use of resources.

The article “Performance of the ECR Ion Source of CERN's Heavy Ion Injector” by M. P. Bougarel et al., presented at the 12th International Workshop on ECR Ion Sources, Apr. 25-27, 1995, in Riken, Japan, describes, among other things, the “afterglow mode” of an ECR ion source. This involves the ECR ion source being operated in pulsed mode (i.e., the microwave irradiation is not used continuously but is pulsed). It was observed that, in this mode, after a microwave beam pulse has been switched off (e.g., in the afterglow phase) a short ion pulse with a high current intensity is emitted from the ECR ion source.

The article “HIMAC and Medical Accelerator Projects in Japan,” by S. Yamada et al., in the Proceedings of 1st Asian Particle Accelerator Conference (APAC 98), Tsukuba, Japan, Mar. 23-27, 1998, p. 885, describes how the ion source in the system was operated with krypton ions or iron ions in the “afterglow mode”.

### SUMMARY AND DESCRIPTION

The present embodiments may obviate one or more of the drawbacks or limitations in the related art. For example, in one embodiment, a particle therapy system with an ion source, in which high beam intensities are possible, having small dimensions is provided.

The particle therapy installation or system according to the present embodiments includes an ECR ion source for generating charged ions that are accelerated to an energy used for irradiation in an accelerator unit that is arranged downstream of the ECR ion source. The magnetic fields of the ECR ion source are attuned to operation of the ECR ion source with lightweight ions (e.g., ions with a nuclear charge less than or equal to 10). The ECR ion source is operated in the afterglow mode, in which an afterglow beam pulse is emitted from the ECR ion source after a microwave resonance pulse has been turned off. The afterglow beam pulse has a higher current intensity than the current intensity of the ion current that is emitted from the ECR ion source during the use of the microwave resonance pulse.

The high current intensity of ions from the ECR ion source that is generated during an afterglow beam pulse is used. In this way, the ECR ion source may be smaller in dimension overall and thus more cost-effective, since the current intensity used for irradiation is provided by the higher current intensity of the afterglow beam pulse.

The magnetic fields of the ECR ion source are dimensioned such that the ECR ion source may also be operated using lightweight ions. Such an ECR ion source may be operated with carbon ions and/or oxygen ions, for example. With carbon ions, it may be advantageous for the ECR ion source to be operated in pulsed mode in the afterglow mode, since fewer deposits will appear on the walls of the ECR ion source in pulsed mode than in a continuous operating mode of the ECR ion source.

The accelerator unit that is arranged downstream may include a linear accelerator that is operated such that the timing of the linear accelerator is attuned to the timing of the ECR ion source. The timing of the linear accelerator is attuned to the timing of the ECR ion source such that at least part of the ion current emitted from the ECR ion source during the afterglow beam pulse is accelerated by the accelerator unit that is arranged downstream.

In one embodiment, only those ions that have been generated by the afterglow beam pulse may be used for further acceleration. The duration of an afterglow beam pulse may be long enough for the ions generated by the afterglow beam pulse to, themselves, feed the accelerator unit that is arranged downstream.

Unlike continuously operating ECR-ion sources, the particle therapy system of the present embodiments does not use any special elements inserted between the ECR ion source and the downstream linear accelerator in cases where the ECR ion source is operating in the continuous mode to form a beam pulse (e.g., a “chopper”). The linear accelerator that is arranged downstream may include a radio frequency quadrupole (RFQ) accelerator, for example.

The ECR ion source may include a system of magnets that generates an axial magnetic field in the ECR ion source. In one embodiment, the system of magnets includes at least one permanent magnet and at least one variably adjustable electromagnet in addition to the permanent magnet.

In addition to the system of magnets for the axial magnetic field, the ECR ion source includes a system of magnets for the radial magnetic field. For example, a hexapolar magnetic field may be generated with the system of magnets for the radial magnetic field. In addition to or as an alternative embodiment, the system of magnets for the radial magnetic field likewise includes at least one permanent magnet and at least one variably adjustable electromagnet, in addition to the permanent magnet.

The axial magnetic field or the radial magnetic field of the ECR ion source may be calibrated and adjusted in a simple and quick manner using the variably adjustable electromagnet or electromagnets. If, for example, an ECR ion source is to be operated with carbon ions and oxygen ions, the axial magnetic field or the radial magnetic field may be quickly and simply adjusted to the type of ions used using the variably adjustable electromagnet or electromagnets. A fine adjustment of the ECR ion source may also be effected in a simple manner. Unlike ECR ion sources with an axial magnetic field or a radial magnetic field generated entirely by electromagnets, the electromagnet or electromagnets used in combination with one or a plurality of permanent magnets may be designed to be more cost effective and more energy-efficient.

### BRIEF DESCRIPTION OF THE DRAWINGS

FIG. 1 a schematic overview of a particle therapy system,

FIG. 2 a longitudinal section through an ECR ion source,

FIG. 3 a schematic overview of the first section of the accelerator in a particle therapy system, and

FIG. 4 a diagram to show the timing of the ECR ion source and the linear accelerator unit that is arranged downstream.

### DETAILED DESCRIPTION OF THE DRAWINGS

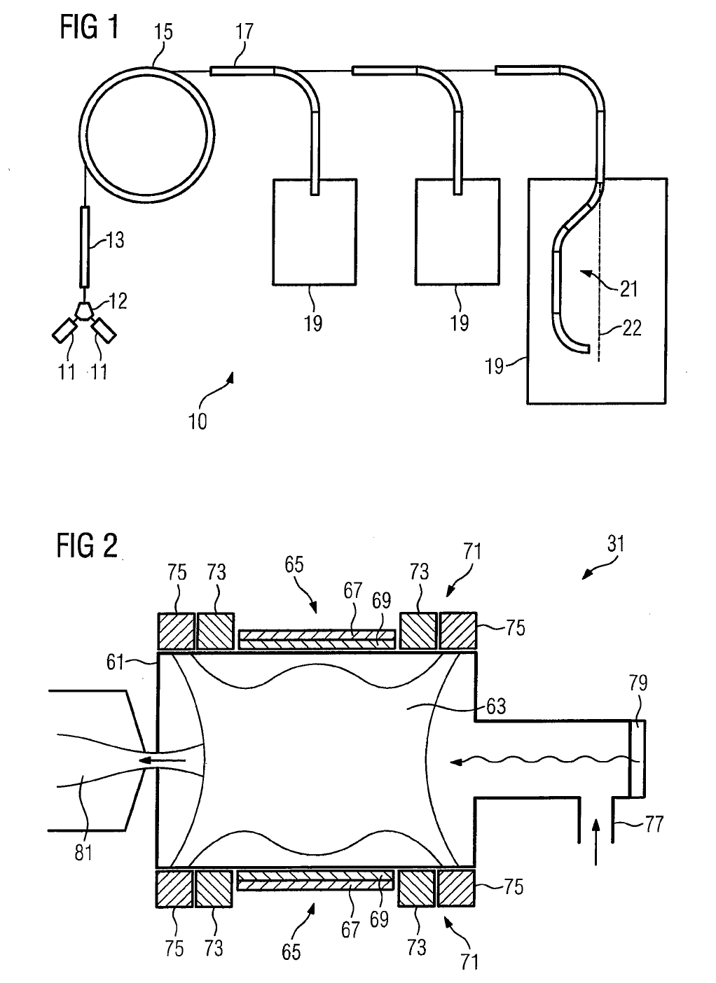

FIG. 1 shows a schematic view of a particle therapy system 10. In a particle therapy system 10, irradiation of a body (e.g., a tumorous tissue) is achieved with a particle beam.

The particles used may be ions such as, for example, protons, pions, helium ions, carbon ions or other types of ions. Such particles may be generated in a particle source 11. If, as shown in FIG. 1, there are two particle sources 11 that generate two different types of ions, one of the two types of ions may be switched to the other type of ions within a short period of time. For this purpose, a switching magnet 12, for example, is used. The switching magnet is arranged between the ion sources 11 and a pre-accelerator 13. Thus, for example, the particle therapy system 10 may be operated with protons and carbon ions at the same time.

The ions generated by one of the ion sources 11 and optionally selected using the switching magnet 12 are accelerated in the pre-accelerator 13 to a first energy level. The pre-accelerator 13 is, for example, a linear accelerator (LINAC). The particles are subsequently fed into an accelerator 15 (e.g., a synchrotron or cyclotron). In the accelerator 15, the particles are accelerated to high energies used for irradiation. After the particles leave the accelerator 15, a high energy beam transport system 17 directs the particle beam into an irradiation chamber or a plurality of irradiation chambers 19. In an irradiation chamber 19, the accelerated particles are directed onto a body that is to be irradiated. The accelerated particles may be directed onto a body that is to be irradiated from a fixed direction (e.g., in “fixed beam” chambers) or from different directions via a moveable gantry 21 that is rotatable around an axis 22.

The design of the particle therapy system 10 that is shown in FIG. 1 is known in the prior art and is typical of many particle therapy systems, but may also deviate therefrom.

The embodiments that are described with the aid of the drawings set out hereafter may be used in the particle therapy system 10 illustrated in FIG. 1.

FIG. 2 shows a longitudinal section through an ECR ion source 31.

The ECR ion source 31 includes a chamber 61, in which a plasma 63 that is to be heated is located. A plurality of systems of magnets are arranged around the chamber 61. A first system of magnets 65 generates a radial magnetic field. The first system of magnets 65 is shown in FIG. 2 (indicated schematically) as a system of magnets that includes at least both a permanent magnet 69 and an electromagnet 67 for the radial magnetic field, with the permanent magnet 69 generating a static basic component of the radial magnetic field, and the electromagnet 67 superimposing further radial components of the magnetic field on the static basic component.

A second system of magnets 71 generates an axial magnetic field. The second system of magnets 71 includes at least one permanent magnet 73 and at least one electromagnet 75 (indicated schematically), with the permanent magnet 73 generating a static basic component of the axial magnetic field, and the electromagnet 75 superimposing further axial magnetic field components on the static basic component.

The electromagnet 67 for the radial magnetic field and/or the electromagnet 75 for the axial magnetic field may be selected and set using a control unit. As a result, variable adjustments (e.g., fine adjustments) may be made to the axial magnetic field or the radial magnetic field in a simple manner. The magnetic field may also be easily adjusted in this way if the ECR ion source 31 is to be operated with another type of ions. Since at least part of the axial magnetic field component or at least part of the radial magnetic field component is generated by the permanent magnets 69, 73, the electromagnets 67, 75 may be designed accordingly with smaller dimensions and thus be operated more favorably.

On one side of the chamber 61 is a gas inlet 77, through which molecules may be injected into the chamber 61. Additionally, the ECR ion source 31 includes a device 79 for generating microwave irradiation. With the aid of the microwave irradiation, the plasma electrons may be accelerated when there is appropriate resonant tuning of the frequency of the microwave irradiation, and thus, the plasma 63 may be heated. The resulting ions are emitted from the ECR ion source 31 in an ion current 81.

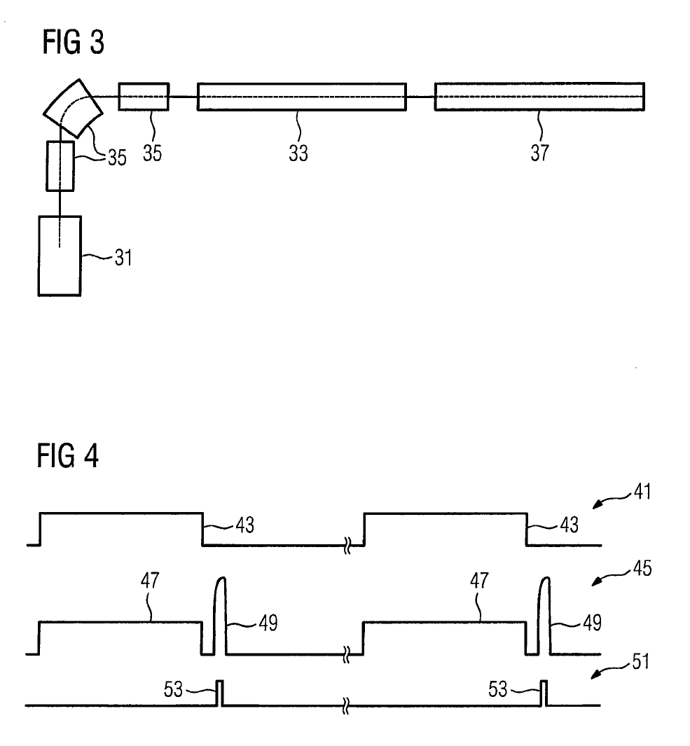

FIG. 3 shows a schematic view of the ECR ion source and an accelerator unit that is arranged downstream.

Downstream of the ECR ion source 31 is an RFQ accelerator 33, which accelerates ion pulses that enter the RFQ accelerator 33. The RFQ accelerator 33 may include an IH drift tube linear accelerator 37 arranged downstream. Since the ECR ion source 31 is operated in an afterglow mode, as described below with the aid of FIG. 4, the RFQ accelerator 33 is arranged downstream of the ECR ion source 31 such that no element is arranged between the ECR ion source 31 and the RFQ accelerator 33 to “chop” the ion current that is emitted from the ECR ion source 31 (e.g., a macro pulse chopper may otherwise be arranged between an ion source and an accelerator). However, further beam-forming or beam-measuring elements 35 such as, for example, focusing and/or defocusing magnets (e.g., solenoids, quadrupole magnets, spectrometer magnets or beam diagnosis devices) are arranged between the ECR ion source 31 and the RFQ accelerator 33.

A further accelerator unit, such as a synchrotron, for example, may be arranged downstream of the linear accelerator unit shown in FIG. 3.

FIG. 4 shows, with the aid of schematic diagrams, the temporal tuning of the timing between the ECR ion source and the RFQ accelerator that is arranged downstream.

The first line 41 of FIG. 4 shows the switching on and off of the microwave irradiation in the ECR ion source 31, the microwave irradiation being used in pulsed mode. Microwave irradiation is used, for example, as a microwave irradiation pulse 43 of 10 ms duration and is subsequently paused. If the ECR ion source 31 is operated at a frequency of 5 Hz, for example, the application of the microwave irradiation pulses 43 that have a duration of 10 ms is effected at intervals of 200 ms.

As long as a microwave irradiation pulse 43 is applied, an ion current corresponding to the microwave irradiation pulse is emitted from the ECR ion source 31 (shown in the second line 45 of FIG. 4 in idealized form with a rectangular ion current pulse line 47). After the microwave impulse pulse 43 has been terminated, an ion current pulse of short duration is subsequently emitted from the ECR ion source 31 at a high beam intensity. The ion current pulse emitted in the afterglow phase is an afterglow beam pulse 49. The afterglow beam pulse is characterized by a short duration of a maximum of a few milliseconds and by a high current intensity (e.g., compared with the ion current intensity of the ion current during the microwave irradiation pulse 43).

The third line 51 shows the timing of the RFQ accelerator 33 in schematic form, the RFQ accelerator 33 being temporally tuned such that a HF pulse 53 of the RFQ accelerator 33 (e.g., with a pulse length of 300 μs) falls in the phase of the afterglow impulse pulse 49. In this way, the RFQ accelerator 33 accelerates those ions that have been generated by the afterglow beam pulse 49 to a higher energy.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A particle therapy system comprising:
an ECR ion source for the production of charged ions; and
an accelerator unit that is arranged downstream of the ECR ion source, the accelerator unit for accelerating the charged ions to an energy required for irradiation,
wherein magnetic fields of the ECR ion source are attuned to the operation of the ECR ion source for lightweight ions such that the ECR ion source is operable to be operated in an afterglow mode, in which, an afterglow beam pulse is emitted from the ECR ion source after a microwave resonance pulse has been switched off, and
wherein the current intensity of the afterglow beam pulse is higher than a current that is emitted from the ECR ion source during the application of the microwave resonance pulse.

2. The particle therapy system as claimed in claim 1, wherein the ECR ion source is configured to operate with carbon ions, oxygen ions or carbon ions and oxygen ions.

3. The particle therapy system as claimed in claim 1, wherein the accelerator unit that is arranged downstream of the ECR ion source includes a linear accelerator, and
wherein the timing of the linear accelerator is attuned to the ECR ion source operating in the afterglow mode such that at least part of the ion current emitted from the ECR ion source during the afterglow beam pulse is accelerated by the linear accelerator.

4. The particle therapy system as claimed in claim 3, wherein the linear accelerator is arranged downstream of the ECR ion source without an element that forms a beam pulse being arranged between the ECR ion source and the linear accelerator.

5. The particle therapy system as claimed in claim 1, wherein the ECR ion source comprises a first system of magnets for generating an axial magnetic field, and
wherein the first system of magnets comprises a permanent magnet and a variably adjustable electromagnet.

6. The particle therapy system as claimed in claim 5, wherein the ECR ion source comprises a second system of magnets for generating a radial magnetic field, and
wherein the second system of magnets comprises a permanent magnet and a variably adjustable electromagnet.

7. The particle therapy system as claimed in claim 2, wherein the accelerator unit that is arranged downstream of the ECR ion source includes a linear accelerator, and
wherein the timing of the linear accelerator is attuned to the ECR ion source operating in the afterglow mode such that at least part of the ion current emitted from the ECR ion source during the afterglow beam pulse is accelerated by the linear accelerator.

8. The particle therapy system as claimed in claim 2, wherein the ECR ion source comprises a system of magnets for generating an axial magnetic field, and
wherein the system of magnets comprises a permanent magnet and a variably adjustable electromagnet.

9. The particle therapy system as claimed in claim 3, wherein the ECR ion source comprises a system of magnets for generating an axial magnetic field, and
wherein the system of magnets comprises a permanent magnet and a variably adjustable electromagnet.

10. The particle therapy system as claimed in claim 4, wherein the ECR ion source comprises a system of magnets for generating an axial magnetic field, and
wherein the system of magnets comprises a permanent magnet and a variably adjustable electromagnet.

11. The particle therapy system as claimed in claim 1, wherein the ECR ion source comprises a system of magnets for generating a radial magnetic field, and
wherein the system of magnets comprises a permanent magnet and a variably adjustable electromagnet.

12. The particle therapy system as claimed in claim 2, wherein the ECR ion source comprises a system of magnets for generating a radial magnetic field, and
wherein the system of magnets comprises a permanent magnet and a variably adjustable electromagnet.

13. The particle therapy system as claimed in claim 3, wherein the ECR ion source comprises a system of magnets for generating a radial magnetic field, and
wherein the system of magnets comprises a permanent magnet and a variably adjustable electromagnet.

14. The particle therapy system as claimed in claim 4, wherein the ECR ion source comprises a system of magnets for generating a radial magnetic field, and
wherein the system of magnets comprises a permanent magnet and a variably adjustable electromagnet.

15. The particle therapy system as claimed in claim 3, wherein the accelerator unit is an RFQ accelerator.

16. The particle therapy system as claimed in claim 4, wherein the accelerator unit is an RFQ accelerator.
